(19) United States
(12) Patent Application Publication
(10) Pub. No.: US 2007/0072915 A1
(43) Pub. Date: Mar. 29, 2007

(54) Novel phenoxyacetamide derivative and use thereof for preparation of diphenylamines

(76) Inventors: Claude Lardy, Lyon (FR); Bernard Boudet, Pithiviers (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/580,546

(22) PCT Filed: Nov. 5, 2004
(86) PCT No.: PCT/EP04/12541

§ 371 Date: May 26, 2006

(30) Foreign Application Priority Data
Nov. 27, 2003 (FR) 0313952

(51) Int. Cl. A61K 31/44; A61K 31/4172; A61K 31/277; A61K 31/22; A61K 31/165

(52) U.S. Cl. 514/350; 514/521; 514/621; 514/534; 558/410; 562/450; 564/167; 564/431; 546/350; 514/400; 548/335.5

(57) ABSTRACT

The present invention relates to novel phenoxyacetamide derivatives, to a process for the preparation thereof and to the use thereof for the preparation of diphenylamines.

The present invention relates to novel phenoxyacetamide derivatives and to the use thereof for the preparation of diphenylamines.

Antioxidant diphenylamines, especially of therapeutic interest, have been described previously in FR 2 815 030 or WO 02/28820.

The synthesis of diphenylamines has previously been described by coupling an aromatic halide with an aromatic amine in the presence of palladium derivatives (Prashad M. et al., J. Org. Chem. (2000) 65, 2612-1614; Sadighi J. P., Harris M. C., Buchwald S. L., Tetrahedron Lett. (1998) 39, 5327-5330; Yang B. H., Buchwald S. L., J. Organomet. Chem. (1999) 576,125-146; Hartig J. F., Angew. Chem. Int. Ed. Engl. (1998) 37, 2046-2067).

Other methods use coupling via arylmagnesium derivatives (Sapountzis I., Knockel P., J. Am. Chem. Soc. (2002) 124, 9390).

However, the processes described have low yields, use expensive catalysts, such as derivatives of palladium and its ligands, such as BINAP, and the traces of which are difficult to remove from the products obtained, or require the use of moisture-sensitive reagents.

The Smiles rearrangement, described especially by Levi et al., J. Chem. Soc., 1931, 3264, makes it possible to prepare diphenylamines readily, in good yields, while avoiding the problems mentioned above.

However, the preparation of the diphenylamines discussed above by the Smiles rearrangement requires specific phenoxyacetamide intermediates, which have not been described hitherto.

The inventors have now synthesized the corresponding phenoxyacetamide derivatives that allow the desired diphenylamine derivatives to be prepared.

Consequently, the present invention relates to the phenoxyacetamides of the formula (I) 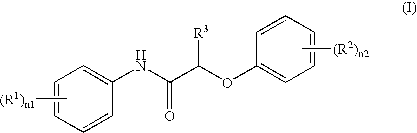
to the use thereof for the preparation of the corresponding diphenylamines of the formula (II) 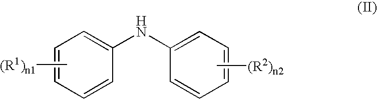
and to the process for the preparation of the said derivatives of the fornula (I).

In the general formula (I):

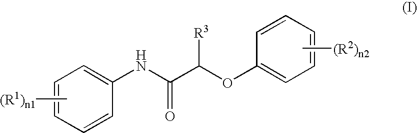

R¹, which may be identical or different, are chosen independently from -Hal, —O-Alk, —N(Alk)₂, —NH—C(═O)-Alk, —O-C-Hal₃, —NO₂, —NH₂, —NHAlk, —C(═O)Hal, C(═O)OAlk, —OH, —C(═O)—NH₂, —C(═O)—NHAlk, —C(═O)—N(Alk)₂, —NH—(C═O)—OAlk, —H, —CN, -Alk, —C(═O)Alk, —NAlk-CO—OAlk and —NAlk₂;

n1=integer between 1 and 5;

R², which may be identical or different, are chosen independently from —X—(C═O)—Y-(A)n and —CN;

n2 =integer between 1 and 5;

n=0 whenY=Hal; n=1 when Y═O or n=2 when Y═N;

X=bond or -Alk-;

Y═—O—, —N— or Hal;

A=—H, -Alk, -Alk-Ar, -Alk-Het, —Ar or -Het, Ar and Het being optionally substituted by Hal or Het;

R³═—H, -Alk, —NAlk₂, —NHAlk or —NH₂ and the addition salts thereof,

with the exception of the compounds for which:
R²═—CN in position 4 (4-CN), n2=1, and

R³═H, n1=2 and R¹=(2-NHMe, 5-NO2 ); or

R³=Me, n1=1 and R¹=-4-(NH—(C═O)O—Me);
R²=4-(—COOH), n2=1, and

R³═—(CH₂)₁₅-Me, n1=4 and R¹=(2-OH,3,5-diCl,4-Et)

R³═—H, n1=4 and R¹=(2-OH,3,5-diCl,4-Me)
R²=4-(—COOMe), n2=1, n1=1 and

—R³═H, R¹═H, 2-(C(═O)—CH₃)
Preferably, R¹=-Hal, —O-Alk, —N(Alk)₂, —NH—C(═O)-Alk, —O—C-Hal₃ or —NO₂.
Preferably, n1=1.
Preferably, R²═—(C═O)═NH₂, —COOH, —COHal, —(C═O)—Oalk or —CN, —(C═O)—NH-Het, —(C═O)NH-Alk-Het, -Alk-(C═O)—NH-Phe, -Alk-(C═O)—NH-Het, -Alk-(C═O)—NH-Phe-Hal or -Alk-(C═O)—NH-Phe-Het.
Preferably, n2=1.
Preferably, R³═—H or -Alk.
Even more preferably, —R¹═—F, —Cl, —O-Me, —NMe₂, —NH—C(═O)Me, —O—CF₃ or —NO₂.
Even more preferably, n1=1.
Even more preferably, R²═—CN, —COOH, —COCl, —(C═O)OMe, —(C═O)—OEt, —(C═O)—NH₂, —CH₂—C(═O)—NH-Py, —(C═O)—NH-Py, —(C═O)—NH—(CH₂)₃-lm, —CH₂—(C═O)—NH-Phe, —CH₂—(C═O)—NH-Phe-F or —CH₂(C═O)—NH-Phe-Morph.
Even more preferably, n2=1.
Even more preferably, R³═—H or Et.

In a particularly advantageous manner, the following compounds are preferred:
4-{2-[(4-methoxyphenyl)amino]-2-oxoethoxy}-N-pyridin-3-ylbenzamide
methyl 4-{2-[(4-methoxyphenyl)amino]-2-oxoethoxy}benzoate
4-{2-[(4-methoxyphenyl)amino]-2-oxoethoxy}benzoic acid
4-{2-[(4-methoxyphenyl)amino]-2-oxoethoxy}benzoyl chloride
2-(4-{2-[(4-fluorophenyl)amino]-2-oxoethyl}phenoxy}-N-2-methoxyphenyl)acetamide
2-(4-{2-[(4-methoxyphenyl)amino]-2-oxoethoxy}phenyl}-N-4-morpholin-4-ylphenyl)-acetamide
2-[4-(2-anilino-2-oxoethyl)phenoxy]-N-(4-methoxyphenyl)acetamide
2-(4-{2-[(4-methoxyphenyl)amino]-2-oxoethoxy}phenyl)-N-pyridin-3-yl-ethanamide
ethyl 4-{2-[(4-methoxyphenylamino]-2-oxoethoxy}benzoate
N-[3-(1H-imidazol-1-yl)propyl]-4-{2-[(methoxyphenyl)amino]-2-oxoethoxy}benzamide
methyl 4-{2-[(4-dimethylaminophenyl)amino]-2-oxoethoxy}benzoate
methyl 4-{2-[(4-N-acetylaminophenyl)amino]-2-oxoethoxy}benzoate
4-(2-oxo-2-{[4-(trifluoromethoxy)phenyl]amino}ethoxy)benzamide
2-(4-cyanophenoxy-N-[4-(trifluoromethoxy)phenyl]acetamide
4-{2-[(4-fluorophenyl)amino]-2-oxoethoxy}benzamide
4-{2-[(4-nitrophenyl)amino]-2-oxoethoxy}(benzamide
methyl 4-{2-[(3-chlorophenyl)amino]-2-oxoethoxy}benzoate
methyl 4-{2-[(4-fluorophenyl)amino]-2-oxoethoxy}benzoate
methyl 4-(1-{[(4-fluorophenyl)amino]carbonyl}propoxy)benzoate
   and the addition salts thereof.

Preferably, the following compounds do not form part of the invention:
R¹=4—(C═O)—OEt, n1=1, R²=4-(-CH₂—(C═O)—OEt), n2=1 and R³=-Me;
R²═—CN in position 4 (4-CN), n2=1, and

R³═H, n1=1 and R¹=2-, 4-F, 2-Cl, 3-Cl, 4-Cl, 4-Br, 3-OMe, 4-OMe; 4-OEt, 2-Me; 3-Me or 4-Me; or

R³═H, n1=2 and R¹=(2,3-diCl); (2,4-diCl); (3,4-diCl); (3,5-diCl); (3-Cl, 4-F); (2-NO₂, 4-OEt); or (2,3-diMe); (2,5-diMe); (2,6-diMe); (3,4-diMe); (3,5-diMe); (2-Me; 3-Cl); (2-Me, 4-Cl); (3-Cl, 4-Me); (3-Me, 4-Br).

R³=Me, n1=1 and R¹═—(C═O)—OEt;

R³═H, n1=3 and R¹=2,4,6-triMe;
R²=4-(-(C═O)N(H)-Et-Phe), n2=1, R³═H, and

n1=1 and R¹=4-Cl, 4-F, 2-Cl, 2-OEt, 2-nBu, 2-iPr, 2-Et, 4-Et, 4-Me, or

n1=2 and R¹=(3,5-diCl); (2-Me, 3-Cl); (2,6-diEt); (3-Me, 5-Cl); (2,3-diMe); (2,4-diMe);
R²=4-(-COOH), n2=1, and

R³═H, n1=1 and R¹=4-OMe,
R²=4-(—COOMe), n2=1, n1=1 and

R³═H, R¹=4-Cl or 4-COOEt,

R³=Me, R¹=4-COOEt.

According to the present invention, the term “Alk” means a linear or branched aliphatic hydrocarbon-based group containing from 1 to 20 carbon atoms in the chain, the said “Alk” substituent possibly comprising one or more unsaturations; preferably, “Alk” represents an alkyl, alkenyl or alkynyl group.

Typical examples of alkyl groups include methyl, ethyl, propyl, butyl, pentyl and hexyl and each of the corresponding iso, sec and tert derivatives thereof.

Typical examples of alkenyl groups include ethenyl, propenyl, butenyl, pentenyl, hexenyl, heptenyl and octenyl, and each of the corresponding iso, sec and tert derivatives thereof.

Typical examples of alkynyl include ethynyl, propynyl, butynyl, pentynyl and hexynyl, and each of the corresponding iso, sec and tert derivatives thereof.

Preferably, “Alk” denotes methyl, ethyl, propyl or isopropyl groups.

The term “Alk” also includes the corresponding alkylene, alkenylene and alkynylene derivatives.

The term “Ar” denotes a monocyclic or polycyclic, preferably bicyclic, aromatic, hydrocarbon-based or heteroaryl ring system containing from 6 to 14 carbon atoms and preferably between 6 and 10 carbon atoms.

Typical examples of aryl groups include phenyl and naphthyl.

The term “aromatic” denotes a cyclically conjugated hydrocarbon-based aryl or heteroaryl, which obeys Huckel's rule and/or has a delocalization stability that is substantially higher than that of a hypothetical localized structure.

The term “Hal” denotes fluoro, chloro, bromo or iodo substituents. Fluoro and chloro are preferred.

The term “Het” denotes a monocyclic or polycyclic, preferably bicyclic, aromatic heteroaryl or non-aromatic heterocyclic ring system, containing between 5 and 14 carbon atoms and preferably between 5 and 10 carbon atoms, in which one or more atoms in the ring system are one or more hetero elements, such as nitrogen, oxygen or sulfur. Preferably, the ring contains 5 or 6 atoms.

The preferred heteroaryl groups include furyl, pyranyl, benzofuranyl, chromenyl, xanthenyl, pyrrolyl, imidazolyl, pyrazolyl, thiazolyl, oxazolyl, pyridyl, pyrazinyl, pyrimidinyl, pyridazinyl, indolyl, indazolyl, purinyl, quinolizinyl, quinolyl, phthalazinyl, naphthyridinyl, quinoxalinyl, quinazolinyl, cinnolinyl, thienyl, isothiazolyl, furazanyl, 1,2,4-thiadiazolyl, imidazo[1,2-a]pyridyl, imidazo[2,1-b]thiazolyl, benzofurazanyl, azaindolyl, benzimidazolyl, benzothienyl, thienopyridyl, thienopyrimidinyl, pyrrolopyridyl, imidazopyridyl, benzazaindolyl, 1,2,4-triazinyl, benzthiazolyl, indolizinyl, isoxazolyl, isoquinolyl, isothiazolyl, oxadiazolyl, quinolyl, 1,3,4-thiadiazolyl, thiazolyl, thienyl and triazolyl. Non-aromatic heterocyclic substituents especially include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl and tetrahydrothiopyranyl, and the like.

The term “Phe” denotes a phenyl group.

The term “Py” denotes a pyridyl substituent.

The term “Morph” denotes a morpholinyl substituent.

The term “addition salts” refers to the organic or mineral base-addition or acid-addition salts of the compounds of the present invention. These salts can be prepared in situ during the final isolation and purification of the compounds. In particular, the acid-addition salts can be prepared by separately reacting the purified compound in its purified form with an organic or mineral acid and isolating the salt thus formed. Among the examples of acid-addition salts are the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, sulfamates, malonates, salicylates, propionates, methylenebis-b-hydroxynaphthoates, gentisic acid, isethionates, di-p-toluoyltartrates, methanesulfonates, ethanesulfonates, benzenesulfonates, p-toluenesulfonates, cyclohexyl sulfamates and quinateslaurylsulfonate, and analogues. (See for example S. M. Berge et al. “Pharmaceutical Salts” J. Pharm. Sci, 66: pp.1-19 (1977), incorporated herein by reference). The acid-addition salts can also be prepared by separately reacting the purified compound in its acidic form with an organic or mineral base and isolating the salt thus formed. The acid-addition salts include amines salts and metal salts. The suitable metal salts include the sodium, potassium, calcium, barium, zinc, magnesium and aluminium salts. The sodium and potassium salts are preferred. The suitable mineral base-addition salts are prepared from metallic bases including sodium hydride, sodium hydroxide, potassium hydroxide, calcium hydroxide, aluminium hydroxide, lithium hydroxide, magnesium hydroxide and zinc hydroxide. The suitable amine base-addition salts are prepared from amines whose basicity is sufficient to form a stable salt, and preferably include the amines that are often used in medicinal chemistry on account of their low toxicity and their acceptability for medical use: ammonia, ethylenediamine, N-methylglucamine, lysine, arginine, ornithine, choline, N,N′-dibenzylethylenediamine, chloroprocaine, diethanolamine, procaine, N-benzyl-phenethylamine, diethylamine, piperazine, tris(hydroxymethyl)aminomethane, tetra-methylammonium hydroxide, triethylamine, dibenzylamine, ephenamine, dihydro-abietylamine, N-ethylpiperidine, benzylamine, tetramethylammonium, tetraethyl-ammonium, methylamine, dimethylamine, trimethylamine, ethylamine, basic amino acids, for example lysine and arginine, and dicyclohexylamine, and analogues.

According to a second aspect, the invention also relates to the use of the phenoxyacetamides of the formula (I) for the preparation of diphenylamines of the formula (II) described above, in which

R¹, which may be identical or different, are chosen independently from -Hal, —O-Alk, —N(Alk)₂, —NH—C(═O)-Alk, —O—C-Hal₃, —NO₂, —NH₂, —NHAlk, —COOH, —C(═O)Hal, —C(═O)OAlk, —OH, —C(═O)—NH₂, —C(═O)—NHAlk, —C(═O)—N(Alk)₂, —NH—(C═O)—OAlk, —H, —CN, -Alk, —C(═O)Alk, —NAlk-CO—OAlk and —NAlk₂;

n1=integer between 1 and 5;

R², which may be identical or different, are chosen independently from —X—(C═O)—Y-(A)ₙ and —CN;

n2=integer between 1 and 5;

n=0 when Y=Hal; n=1 when Y═O or n=2 when Y═N;

X=bond or -Alk-;

Y═—O—, —N— or Hal;

A=-H, -Alk, -Alk-Ar, -Alk-Het, —Ar or -Het, Ar and Het optionally being substituted by Het or Hal

R³═—H, -Alk, —NAlk₂, —NHAlk or —NH₂
and also according to the definitions of R¹, R², R³, n1, n2, x, y, n and A defined above, according to the scheme below:

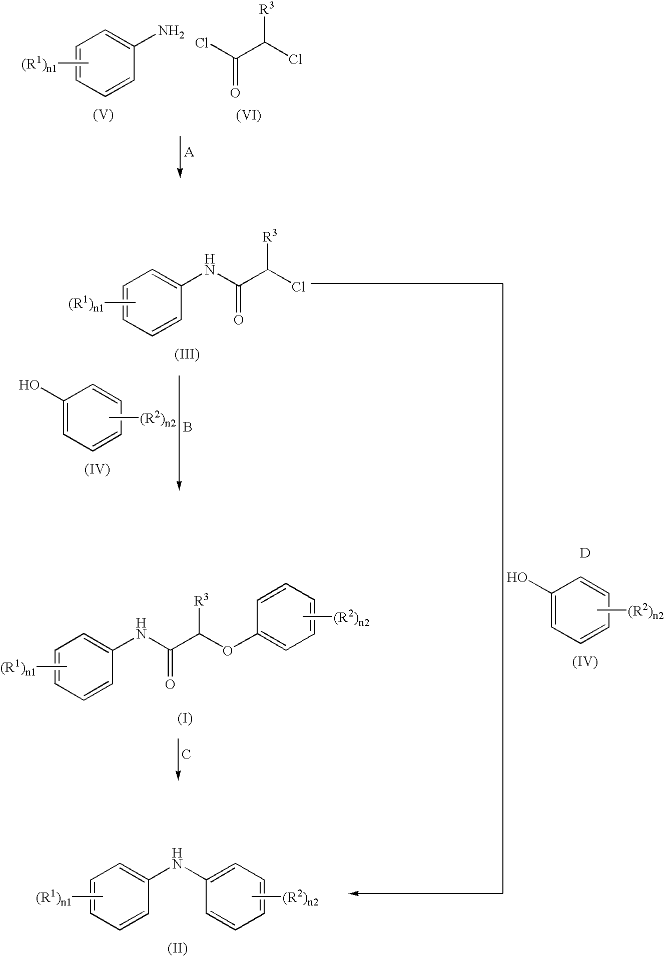

The corresponding process for the preparation of the compounds (II) from the compounds (I) also forms part of the present invention.

According to a first variant, the process for the preparation of the said diphenylamines from the phenoxyacetamides of the invention uses the Smiles rearrangement (Route C). This reaction can be performed by application or adaptation of the methods described by Levy et al., J. Chem. Soc. 1931, 3264, or Evans et al., J. Chem. Soc. 1935, 181; 1936, 329, or alternatively in Tetrahedron Left., 1989, 30, 931-934, WO 92/22522, U.S. Pat. No. 5,475,139, Synthesis, 1977, 31-33 or J. Org. Chem., 1983, 48, 5140-43.

The Smiles rearrangement is an intramolecular nucleophilic aromatic substitution resulting in the migration of an aromatic system from one hetero atom to another. Generally, this reaction is performed with stirring in alkaline medium, optionally heated, if necessary. Preferably, the bases used include strong bases, such as sodium hydroxide, potassium hydroxide or potassium carbonate, and also potassium hydride, sodium hydride, sodium carbonate, sodium hydrogen carbonate, sodium methoxide, sodium ethoxide, sodium phenoxide, calcium hydroxide, aluminium hydroxide, lithium hydroxide, magnesium hydroxide, zinc hydroxide, aqueous ammonia, ethylenediamine, diethylamine, pyrazine, tetramethylammonium hydroxide, triethylamine, etc.

The reaction is generally performed with stirring, at a temperature of between 70 and 160° C. and preferably between 130 and 150° C., for a time that is sufficient to achieve an acceptable degree of reaction, generally between 1 and 10 hours and preferably between 5 and 7 hours. Needless to say, the reaction can also be performed in stages, by heating at different temperatures for shorter times, for example in 2-hour sections, at 70° C., followed by 2 hours at 100° C.

In general, any type of solvent can be used, it being understood that it should not have an adverse effect on the conduct of the reaction or on the reagents used. Examples of suitable solvents especially include aromatic, aliphatic or cyclic hydrocarbons, such as hexane, cyclohexane, benzene, toluene or xylene; amides, such as dimethylformamide; and also ethers, such as diethyl ether or tetrahydrofuran, or alternatively dimethyl sulfoxide or dioxane. Preferably, the reaction is performed in the presence of dimethylformamide.

According to one variant of the process according to the invention, the diphenylamines of the formula (II) can be obtained in situ, from the starting reagents III and IV, without necessarily isolating the intermediate phenoxyacetamides of the formula I obtained (Route D). This allows rapid access to the compounds (II).

The diphenylamines thus prepared can be obtained from the reaction mixture by any conventional means known per se. For example, the compounds can be extracted by distilling off the solvent from the reaction mixture or, if necessary, after distilling off the solvent from the reaction mixture, the residue obtained can be extracted using water and a water-immiscible organic solvent, such as ethyl acetate, ethyl ether, dichloromethane, etc. The recovered organic phases are combined and then washed and dried over a hygroscopic salt, for example Na₂SO₄, before being concentrated under vacuum.

Additionally, the product can, if necessary, also be purified by various techniques, such as recrystallization, reprecipitation or chromatographic techniques, such as a column chromatography or preparative TLC.

If necessary, the addition salts can also be formed from the diphenylamines, by reacting the free base with a suitable acid, via methods known per se. If necessary, the diphenylamines can be generated from the addition salts thereof by treatment with a base, such as aqueous sodium bicarbonate, aqueous ammonia or sodium hydroxide.

According to another aspect, the invention also relates to the process for the preparation of the phenoxyacetamides of the formula (I) from the compounds of the formulae (III) and (IV) according to the reaction scheme according to (B) indicated above.

According to route B, the compounds of the formula (I) according to the invention are prepared from the compounds of the formula (III) and of the formula (IV), in a suitable solvent medium, with stirring.

In general, any type of solvent can be used, it being understood that it should not have an adverse effect on the conduct of the reaction or on the reagents used. Examples of suitable solvents especially include aromatic, aliphatic or cyclic hydrocarbons, such as hexane, cyclohexane, benzene, toluene or xylene; amides, such as dimethylformamide; and also ethers, such as diethyl ether or tetrahydrofuran. The reaction is preferably performed in the presence of dimethylformamide.

The reaction can be performed over a wide range of temperatures, and the precise temperature is not critical according to the invention. In general, it is suitable to perform the reaction at a temperature of between 10 and 150° C. and preferably between room temperature and 130° C. The time required for the reaction may vary considerably as a function of various factors, for example the reaction temperature or the nature of the reagents. However, when the reaction is performed under the conditions recommended above, a time of between half an hour and 6 hours and preferably between 1 to 3 hours is generally sufficient.

This reaction is generally performed in alkaline medium and optionally heated, if necessary. The bases preferably used include strong bases, such as sodium hydroxide, potassium hydroxide or potassium carbonate, and also potassium hydride, sodium hydride, sodium carbonate, sodium hydrogen carbonate, sodium methoxide, sodium ethoxide, sodium phenoxide, calcium hydroxide, aluminium hydroxide, lithium hydroxide, magnesium hydroxide, zinc hydroxide, aqueous ammonia, ethylenediamine, diethylamine, pyrazine, tetramethylammonium hydroxide, triethylamine, etc.

After this reaction, the compounds of the general formula (I) can be extracted and/or purified by adaptation or application of the methods described above.

Generally, the compounds of the general formulae (III) and (IV) are commercially available.

If necessary, the compounds of the general formulae (III) and (IV) can be synthesized via methods that are known per se.

For example, the compound of the general formula (III) can be obtained from the compounds of the general formulae (V) and (VI) according to route A of the scheme indicated above.

This reaction is generally performed for a period of from 1 to 16 hours and preferably 3 to 6 hours, with stirring, in a solvent, such as dichloromethane or toluene, at a temperature of between 150° C. and the boiling point of the solvent and preferably between 20 and 30° C., in the presence of a base, such as triethylamine, pyridine or dimethylaminophenyl, or any other method by application or adaptation of methods that are known per se. In the text hereinabove and hereinbelow, the reactions according to the invention can be generally performed by application or adaptation of the methods described in the literature, for example those described by Larock in Comprehensive Organic Transformation, VCH Pub, 1989.

If necessary, in the reactions discussed above, the functional reactive groups can be protected by means of protecting groups, especially those described by Greene and Wuts in Protective Groups in Organic Chemistry, John Wiley and Sons, 1991, or McOmie in Protective Groups in Organic Chemistry, Plenum Press, 1973.

The compounds of the invention may contain asymmetric centres. These asymmetric centres may independently be of R configuration or of S configuration. The various stereoisomers or racemic mixtures also form part of the present invention.

The compounds of the invention may also exhibit geometrical isomerism or have tautomeric forms. Such isomers also form part of the present invention. Generally, these isomers can be separated from mixtures by application or adaptation of methods that are known per se, for example chromatographic or recrystallization techniques, or they can also be prepared from suitable isomers of their intermediates.

Needless to say, the various preferred embodiments indicated hereinabove or hereinbelow can be taken individually or in combination with each, other.

The examples that follow are given as non-limiting illustrations of the present invention.

Examples 1 to 6 illustrate route B+C; Examples 7 to 18 illustrate route D. Route D was performed by carrying out two successive reactions of “one-pot” type without isolating the intermediate product (I) formed so as to facilitate access to the product (II).

### EXAMPLE 1

### 4-[(4-Methoxyphenyl)amino]-N-pyridin-3-ylbenzamide

### a) 4-{2-[(4-Methoxyphenyl)amino]-2-oxoethoxy}-N-pyridin-3-ylbenzamide

2.26 g (16.34 mmol) of potassium carbonate are added to a solution of 1 g (4.67 mmol) of 4-hydroxy-N-pyridin-3-ylbenzamide in 10 ml of DMF at room temperature, followed by addition of a solution of 0.84 g (4.2 mmol) of 2-chloro-N-(4-methoxyphenyl)acetamide in 7 ml of DMF; the mixture is heated at 110-120° C. for 1.5 hours. After concentrating under vacuum at 60-70° C., the residue is triturated in 25 ml of water. The solid obtained is filtered off, rinsed with water (3×40 ml) and dried under vacuum to give 1.2 g of a khaki-coloured powder.

Yield: 75.7%

NMR:

(DMSO-d₆): 3.72 (3H, s); 4.78 (2H, s); 6.8-8.1 (9H, m); 8.2 (1H, m); 8.3 (1H, m); 8.9 (1H, m); 10.0 (1H, s); 10.31 (1H, s)

### b) 4-[(4-Methoxyphenyl)amino]-N-pyridin-3-ylbenzamide

A mixture of 500 mg (1.32 mmol) of the compound prepared in Example 1a and 549 mg (3.96 mmol) of potassium carbonate in 6 ml of DMF is heated at 140° C. for 5.5 hours.

After cooling, the reaction medium is poured into 25 ml of water and extracted with ethyl acetate (3×20 ml). The combined organic phases are washed with water and dried over Na₂SO₄, before being concentrated under vacuum. 410 mg of a beige-coloured solid are obtained.

Yield: 96.9%

NMR:

(DMSO-d₆): 3.6 (3H, s); 6.9-7.0 (4H, m); 7.1 (2H, m); 7.3 (1H, m); 7.8 (2H, m); 8.15 (1H, m); 8.25 (1H, m), 8.4 (1H, m); 8.9 (1H, m); 10.1 (1H, s).

### EXAMPLE 2

### 4-[(4-Methoxyphenyl)amino]-N-pyridin-3-ylbenzamide

### a) Methyl 4-{2-[(4-methoxyphenyl)amino]-2-oxoethoxy}benzoate

2.26 g (13.4 mmol) of potassium carbonate are added to a solution of 0.71 g (4.67 mmol) of methyl 4-hydroxybenzoate in 10 ml of DMF, followed by addition of a solution of 0.94 g (4.2 mmol) of 2-chloro-N-(4-methoxyphenyl)acetamide in 7 ml of DMF. The mixture is heated for 1.5 hours at 120° C. After cooling, the reaction medium is poured into water and extracted with ethyl acetate. The organic phase is washed with water until neutral and then dried over Na₂SO₄. After concentrating under vacuum, a pasty solid is obtained, which is recrystallized from an ethyl acetate/heptane mixture to give 0.733 g of the expected product.

Yield: 55.5%

NMR:

(DMSO-d₆): 3.7 (3H, s); 3.8 (3H, s); 4.8 (2H, s); 6.9 (2H, m); 7.1 (2H, m); 7.5 (2H, m); 7.9 (2H, m); 10.0 (1H, s)

### b) 4-{2-[(4-Methoxyphenyl)amino]-2-oxoethoxy}benzoic acid

0.256 g (4.57 mmol) of KOH dissolved in 23 ml of water is added to a solution of 0.72 g (2.28 mmol) of the ester prepared in Example 2a in 23 ml of ethanol. The mixture is refluxed for two hours before being concentrated under vacuum. The residue is taken up in 50 ml of water and washed with ethyl ether (2×50 ml). The aqueous phase is acidified with acetic acid to a pH of between 3 and 4. The precipitate formed is filtered off, washed with water and dried under vacuum. 0.48 g of the expected compound is obtained.

Yield: 69.8%

NMR:

(DMSO-d₆): 3.7 (3H, s); 4.75 (2H, s); 6.9 (2H, m); 7.0 (2H, m); 7.5 (2H, m), 7.9 (2H, m); 10.0 (1H, s); 12.65 (1H, broad s).

### c) 4-{2-[(4-Methoxyphenyl)amino]-2-oxoethoxy}benzoyl chloride

A mixture of 0.475 g (1.58 mmol) of the acid prepared in Example 2b and 2 ml of thionyl chloride is refluxed for 2 hours. After cooling, the reaction medium is concentrated under vacuum to give 0.55 g of a pasty solid, which is used without purification.

### d) 4-{2-[(4-Methoxyphenyl)amino]-2-oxoethoxy}-N-pyridin-3-ylbenzamide

A solution of 0.5 g (1.58 mmol) of the acid chloride prepared in Example 2c is added dropwise, while maintaining the temperature at 10° C., to a solution composed of 0.148 g (1.58 mmol) of 3-aminopyridine, 0.44 ml (3.15 mmol) of triethylamine and 1.58 ml of dichloromethane. After stirring for 2 days at room temperature, the reaction medium is poured into water and extracted with dichloromethane. The organic phase is washed with water, dried over Na₂SO₄ and concentrated under vacuum to give 0.523 g of the expected compound, which is identical to that obtained in Example 1a.

Yield: 88%

### e) 4-[(4-Methoxyphenyl)amino]-N-pyridin-3-ylbenzamide

A mixture of 0.5 g (1.32 mmol) of the compound prepared in Example 2d, 0.15 g (2.64 mmol) of KOH and 6 ml of DMF is heated at 145° C. for 5 hours. After cooling, the reaction medium is poured into water (25 ml) and extracted with ethyl acetate (3×20 ml). The combined organic phases are washed with water until neutral and then dried over Na₂SO₄ before being concentrated. 0.4 g of a product identical to that of Example 1b is obtained.

Yield: 95.1%

### EXAMPLES 3 TO 6

The compounds of the formulae I and II were obtained by working as in Example 1.

| Ex | Formula I | Yield | NMR |
|---|---|---|---|
| 3 | 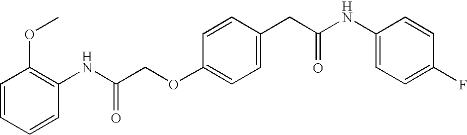 | 68.3% | (DMSO-D6) 3.6(m, 2H) 3.8(m, 3H) 4.7(m, 2H) 7.0(m, 9H) 7.8(dd, J=9.0, 5.3Hz; 2H) 8.1(m, 1H) 9.2(s, 1H) 10.2(s, 1H) |
| 4 | 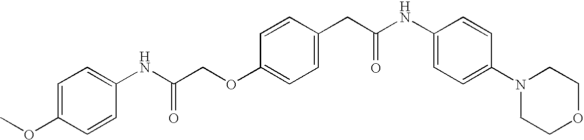 | 81.9% | (DMSO-D6) 3.0(m, 4H) 3.5(s, 2H) 3.7(m, 7H) 4.6(s, 2H) 6.9(m, 6H) 7.2(d, J=8.3Hz, 2H) 7.4(d, J=8.7Hz, 2H) 7.5(d, J=9.0Hz, 2H) 9.9(m, 2H) |
| 5 | 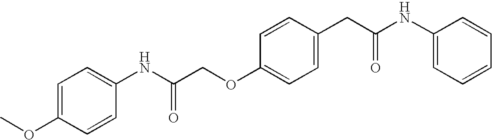 | 40.8% | (DMSO-D6) 3.6(s, 2H) 3.7(m, 3H) 4.6(s, 2H) 7.0(m, 5H) 7.3(m, 4H) 7.6(m, 4H) 9.9(s, 1H) 10.1(s, 1H) |
| 6 | 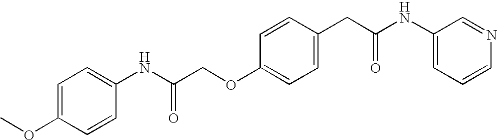 | 9.0% | (DMSO-D6) 3.6(s, 2H) 3.7(s, 3H) 4.6(s, 2H) 6.9(m, 4H) 7.3(m, 3H) 7.5(m, 2H) 8.0(m, 1H) 8.2(m, 1H) 8.7(d, J=2.3Hz, 1H) 9.9(s, 1H) 10.3(s, 1H) |

| Ex | Formula II | Yield | NMR |
|---|---|---|---|
| 3 | 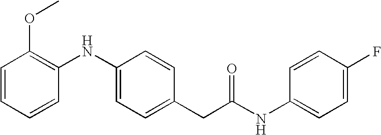 | 15.6% | (DMSO-D6) 3.45-3.68(m, 2H) 3.8(m, 3H) 6.33-10.31(m, 14H) |
| 4 | 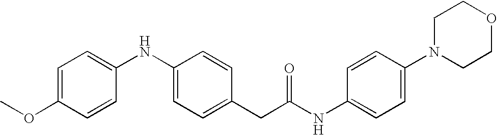 | 49.0% | (DMSO-D6) 3.0(m, 4H) 3.4(s, 2H) 3.7(m, 7H) 6.8(m, 6H) 7.0(d, J=8.7Hz, 2H) 7.1(d, J=8.7Hz, 2H) 7.4(d, J=8.7Hz, 2H) 7.8(s, 1H) 9.8(s, 1H) |
| 5 | 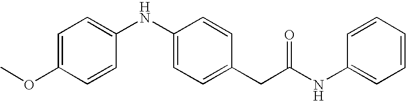 | 34.0% | (DMSO-D6) 3.5(s, 2H) 3.7(s, 3H) 6.8(m, 4H) 7.0(m, 3H) 7.1(d, J=8.3Hz, 2H) 7.3(dd, J=7.9Hz, 2H) 7.6(d, J=8.3Hz, 2H) 7.8(s, 1H) 10.0(s, 1H) |

### EXAMPLE 7

### Ethyl 4-(4-methoxyphenylamino)benzoate via ethyl 4-{2-[(4-methoxyphenyl)amino-2-oxoethoxylbenzoate

A suspension of 400 g (2 mol) of 2-chloro-N-(4-methoxyphenyl)acetamide, 332.4 g (2 mol) of ethyl 4-hydroxybenzoate and 552.8 g (4 mol) of potassium carbonate in 2 l of DMF is refluxed for 6 hours. About 780 ml of DMF are then removed under vacuum and 2.4 l of water are added to the suspension, maintained at 80° C. After cooling, the precipitate formed is filtered off and then washed with water until the filtrate is neutral. After drying, 504.8 g of a beige-coloured powder are obtained.

Yield: 92.9%

m.p.=80° C.

NMR:

(DMSO-d₆): 1.3 (t, J=7.1 Hz, 3 H); 3.7 (s, 3 H); 4.2 (q, J=7.1 Hz, 2 H); 6.9 (m, 4 H); 7.1 (m, 2 H); 7.7 (d, J=8.8 Hz, 2 H); 8.5 (s, 1 H)

### EXAMPLE 8

### 4-[(4-Methoxyphenyl)amino]-N-pyridin-3-ylbenzamide via 4-{(2-[4-methoxyphenyl)amino]-2-oxoethoxy}-N-pyridin-3-ylbenzamide

117.5 g (850 mmol) of potassium carbonate are added at room temperature to a solution of 28 g (131 mmol) of 4-hydroxy-N-pyridin-3-ylbenzamide in 140 ml of DMF. After stirring for 30 minutes at room temperature, a solution of 26.1 g (131 mmol) of 2-chloro-N-(4-methoxyphenyl)acetamide dissolved in 120 ml of DMF is added over 5 minutes. The suspension obtained is heated at 140-145° C. for 9 hours. After cooling, the mixture is distilled under vacuum to remove about 200 ml of solvent, and 140 ml of water are added, followed by extraction with ethyl acetate. The combined organic phases are washed with water and concentrated under vacuum. The residue obtained is stirred for 1 hour in the presence of 80 ml of CH₂Cl₂, filtered and dried in a ventilated oven at 60° C. to give 26.5 g of a beige-coloured powder.

Yield: 63.5%

m.p.=188° C.

NMR:

(DMSO-d₆): 3.7 (s, 3 H); 6.9 (m, 4 H); 7.1 (m, 2 H); 7.3 (dd, J=8.3, 4.7 Hz, 1 H); 7.8 (d, J=8.8 Hz, 2 H); 8.2 (m, 1 H); 8.3 (m, 1 H); 8.4 (s, 1 H); 8.9 (m, 1 H); 10.1 (s, 1 H)

### EXAMPLES 9 TO 17

The compounds of the formula 11 were obtained via the compounds of the formula I, by working as in Example 7 or 8.

| Ex | Formula II | NMR | Yield |
|---|---|---|---|
| 9 | 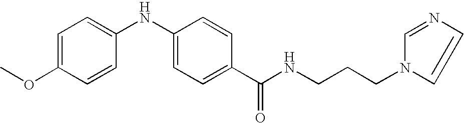 | (DMSO-D6) 1.9(t, J=6.8Hz, 2H) 3.2(m, 2H) 3.7(s, 3H) 4.0(t, J=6.8Hz, 2H) 7.0(m, 8H) 7.7(m, 3H) 8.2(m, 2H) | 70.8% |
| 10 | 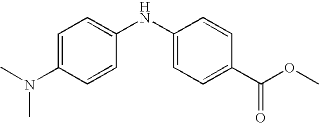 | (DMSO-D6) 2.9(s, 6H) 3.7(s, 3H) 6.8(m, 4H) 7.0(d, J=8.8Hz, 2H) 7.7(d, J=8.8Hz, 2H) 8.4(s, 1H) | 58.4% |
| 11 | 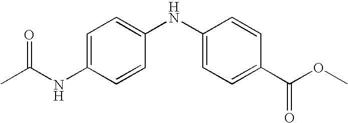 | (DMSO-D6) 2.0(s, 3H) 3.8(s, 3H) 7.0(d, J=8.8Hz, 2H) 7.1(d, J=8.8Hz, 2H) 7.5(d, J=8.8Hz, 2H) 7.8(d, J=8.8Hz, 2H) 8.6(s, 1H) 9.9(s, 1H) | 70.6% |
| 12 | 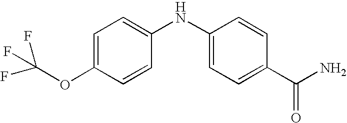 | (DMSO-D6) 7.2(m, 7H) 7.7(m, J=8.0Hz, 3H) 8.7(s, 1H) | 58.4% |
| 13 | 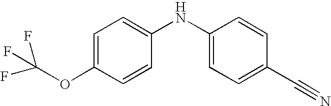 | (DMSO-D6) 7.1(d, J=8.6Hz, 2H) 7.3(m, 4H) 7.6(d, J=8.6Hz, 2H) 9.0(s, 1H) | 75.5% |
| 14 | 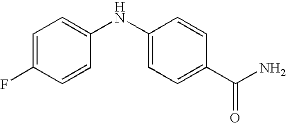 | (DMSO-D6) 7.1(m, 7H) 7.7(m, 3H) 8.5(s, 1H) | 71.8% |
| 15 | 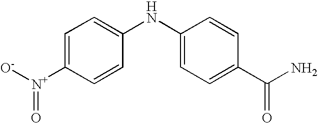 | (DMSO-D6) 7.2(m, 5H) 7.9(m, 3H) 8.1(m, 2H) 9.5(s, 1H) | 15.7% |
| 16 | 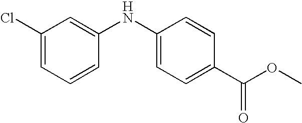 | (DMSO-D6) 3.8(s, 3H) 7.1(m, 6H) 7.8(d, J=8.6Hz, 2H) 8.9(s, 1H) | 27.6% |
| 17 | 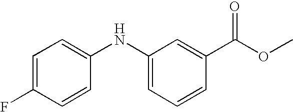 | (DMSO-D6) 3.8(m, 3H) 7.3(m, 8H) 8.4(s, 1H) | 5.1% |

### EXAMPLE 18

### Methyl 4-(1-{[(4-fluorophenyl)amino]carbonyl}propoxy)benzoate and methyl 4-[(4-fluorophenyl)amino]benzoate

3.23 g (15 mmol) of 2-chloro-N-(4-fluorophenyl)butanamide dissolved in 22.5 ml of DMF are added to a mixture of 2.28 g (15 mmol) of methyl 4-hydroxy-benzoate, 13.48 g (98 mmol) of potassium carbonate and 52.5 ml of DMF. After heating at 70° C. for 2 hours, 4.1 g (30 mmol) of potassium carbonate are added, followed by heating at 100° C. for 2 hours. A further amount of K₂CO₃ (6.77 g, 45.4 mmol) is added, followed by heating the reaction medium at 145° C. for 10 hours. After cooling, the reaction medium is poured into 300 ml of water and extracted with ethyl acetate. The organic phase is washed with water and concentrated under vacuum. The residue obtained is purified by chromatography on a column of silica with a (2/1) heptane/ethyl acetate mixture. 1.15 g of methyl 4-(1-{[(4-fluorophenyl)-amino]carbonyl}propoxy)benzoate are obtained as a beige-coloured solid;

Yield: 23%

NMR:

(DMSO-d₆): 1.0 (t, J=7.3 Hz, 3 H); 2.0 (m, 2 H); 3.8 (m, 3 H); 4.8 (t, J=6.2 Hz, 1 H); 7.0 (m, 4 H); 7.7 (m, 4 H); 10.2 (s, 1 H)

and 0.57 g of methyl 4-[(4-fluorophenyl)amino]benzoate as a beige-coloured solid.

Yield: 15.5%

NMR:

(DMSO-d₆): 3.8 (m, 3 H); 6.9-7.7 (m, 8 H); 8.4 (s, 1H)

1. Compounds of the general formula (I) 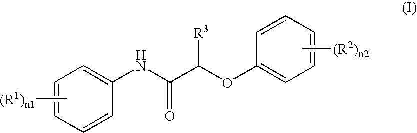
in which:
R¹, which may be identical or different, are chosen independently from -Hal, —O-Alk, —N(Alk)₂, —NH—C(═O)-Alk, —O—C-Hal₃, —NO₂, —NH₂, —NHAlk, —C(═O)Hal, —C(═O)OAlk, —OH, —C(═O)—NH₂, —C(═O)—NHAlk, —C(═O)—N(Alk)₂, —NH—(C═O)—OAlk; —H, —CN, -Alk, —C(═O)Alk, —NAlk-C(O)—O-Alk and —NAlk₂;
n1=integer between 1 and 5;
R², which may be identical or different, are chosen independently from —X—(C═O)—Y-(A)n and —CN;
n2=integer between 1 and 5;
n=0 when Y=Hal; n=1 when Y═O or n=2 when Y═N;
X=bond or -Alk-;
Y═—O—, —N— or Hal;
A=-H, -Alk, -Alk-Ar, -Alk-Het, —Ar or -Het, Ar and Het optionally being substituted by Het or Hal;
R³═—H, -Alk, —NAlk₂, —NHAlk or —NH₂
with the exception of the compounds for which:
R²═—CN in position 4 (4-CN), n2=1,
R³═H, n1=2 and R¹=(2-NHMe, 5-NO₂); or
R³=Me, n1=1 and R¹═—NH—(C═O)—O-Me;
R²=4-(—COOH), n2=1, and
R³═(CH₂)₁₅-Me, n1=4, and R¹=(2-OH,3,5-diCl,4-Et)
R³═H, n1=4, and R¹=(2-OH,3,5-diCl,4-Me)
R²=4-(—COOMe), n2=1, n1=1 and —R³═H, R¹=2-(—C(═O)—CH₃) and also the addition salts thereof.

2. Compounds of the general formula (I) according to claim 1, such that: R¹=-Hal, —O-Alk, —N(Alk)₂, —NH—C(═O)-Alk, —O—C-Hal₃ or —NO₂.

3. Compounds according to claim 1 or 2, in which n1=1.

4. Compounds according to claim 1 claims, in which: R²═—(C═O)—NH₂, —COOH, —COHal, —(C═O)—OAlk, —CN, —(C═O)—NH-Het, —(C═O)—NH-Alk-Het, -Alk-(C═O)—NH-Phe, -Alk-(C═O)—NH-Het, -Alk-(C═O)—NH-Phe-Hal, -Alk-(C═O)—NH-Phe-Het.

5. Compounds according to claim 1, in which n2=1.

6. Compounds according to claim 1, in which R³═—H or -Alk.

7. Compounds according to claim 1 in which R¹═—F, —Cl, —O-Me, —NMe₂, —NH—C(═O)-Me, —O—CF₃ or —NO₂.

8. Compounds according to claim 1, in which: R²═—CN, —COOH, —COCl, —(C═O)—OMe, —(C═O)—OEt, —(C═O)—NH₂, —(C═O)—NH-Py, —CH₂—(C═O)—NH-Py, —(C═O)—NH—(CH₂)₃-lm, —CH₂—(C═O)—NH-Phe, —CH₂—(C═O)—NH-Phe-F or —CH₂—(C═O)—NH-Phe-Morph.

9. Compounds of the general formula (I) according to claim 1, chosen from:
4-{2-[(4-methoxyphenyl)amino]-2-oxoethoxy}-N-pyridin-3-ylbenzamide
methyl 4-{2-[(4-methoxyphenyl)amino]-2-oxoethoxy}benzoate
4-{2-[(4-methoxyphenyl)amino]-2-oxoethoxy}benzoic acid
4-{2-[(4-methoxyphenyl)amino]-2-oxoethoxy}benzoyl chloride
2-(4-{2-[(4-fluorophenyl)amino]-2-oxoethyl}phenoxy}-N-2 methoxyphenyl)acetamide
2-(4-{2-[(4-methoxyphenyl)amino]-2-oxoethoxy}phenyl}-N-4-morpholin-4-ylphenyl)-acetamide
2-[4-(2-anilino-2-oxoethyl)phenoxy]-N-(4-methoxyphenyl)acetamide
2-(4-{2-[(4-methoxyphenyl)amino]-2-oxoethoxy}phenyl)-N-pyridin-3-ylethanamide ethyl
4-{2-[(4-methoxyphenylamino]-2-oxoethoxy}benzoate
N-[3-(1 h-imidazol-1-yl)propyl]-4-{2-[(methoxyphenyl)amino]-2-oxoethoxy}benzamide
methyl 4-{2-[(4-dimethylaminophenyl)amino]-2-oxoethoxy}benzoate methyl 4-{2-[(4-N-acetylaminophenyl)amino]-2-oxoethoxy}benzoate 4-(2-oxo-2-{[4-(trifluoromethoxy)phenyl]amino}ethoxy)benzamide 2-(4-cyanophenoxy-N-[4-(trifluoromethoxy)phenyl]acetamide
4-{2-[(4-fluorophenyl)amino]-2-oxoethoxy}benzamide
4-{2-[(4-Nitrophenyl)amino]-2-oxoethoxy}(benzamide
methyl 4-{2-[(3-chlorophenyl)amino]-2-oxoethoxy}benzoate
methyl 4-{2-[(4-fluorophenyl)amino]-2-oxoethoxy}benzoate methyl
4-(1-{[(4-fluorophenyl)amino]carbonyl}propoxy)benzoate
and also the addition salts thereof.

10. Process for the preparation of the compounds of the general formula 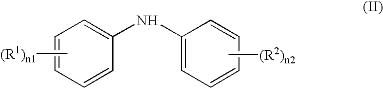
characterized in that a Smiles rearrangement is performed, starting with the compounds of the general formula (1) 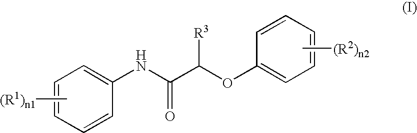
in which, in the general formulae (I) and (II):
R¹, which may be identical or different, are chosen independently from -Hal, —O-Alk, —N(Alk)₂, —NH—C(═O)-Alk, —O—C-Hal₃, —NO₂, —NH2, —NHAlk, —COOH, —C(═O)Hal, —C(═O)OAlk, —OH, —C(═O)—NH2, —C(═O)—NHAlk, —C(═O)—N(Alk)₂, —N—(C═O)—OAlk; —H, —CN, -Alk, —C(═O)Alk, —NAlk-CO—OAlk and —NAlk₂,
n1=integer between 1 and 5;
R², which may be identical or different, are chosen independently from —X—(C═O)—Y-(A)n and —CN;
n2=integer between 1 and 5;
n=O when Y=Hal; n=1 when Y═O or n=2 when Y═N;
X=bond or -Alk-;
Y═—O—, —N— or Hal;
A=-H, -Alk, -Alk-Ar, -Alk-Het, —Ar or -Het, Ar and Het optionally being substituted by Het or Hal;
R³═—H, -Alk, —NAlk₂, —NHAlk or —NH₂.

11. Process according to claim 10, for which, in the general formulae (I) and (II),
R¹=-Hal, —O-Alk, —N(Alk)₂, —NH—C(═O)-Alk, —O—C-Hal₃ or —NO₂;
n1=1;
R²═—(C═O)—NH₂, —COOH, —COHal, —(C═O)—OAlk, —CN, —(C═O)—NH-Het, —(C═O)—NH-Alk-Het, -Alk-(C═O)—NH-Phe, -Alk-(C═O)—NH-Het, -Alk-(C═O)—NH-Phe-Hal or -Alk-(C═O)—NH-Phe-Het,
n2=1;
R³═—H or -Alk.

12. Process according to claim 10 or 11, for which, in the general formulae (I) and (II),
R¹═—F—Cl, —O-Me, —NMe₂, —NH—C(═O)-Me, —O—CF₃ or —NO₂,
N1_{—}1;
R²═—CN, —COOH, —COOl, —(C—O)-Me, —(C═O)—OEt, —(C═O)—NH_{2. —(C═)—NH-Py},
—CH₂—(C+O)—NH-Py, —(C═O)—NH—CH—(CH₂)₃-lm, —CH2-(C═O)—NH-Phe,
—CH₂—(C═O)—NH-Phe-F or —CH₂—(C═O)—NH-Phe-Morph,
n2-1;
R³═—H or Et

13. Process according to claim 10, for which the compounds of the general formula (I) are chosen from:
4-{2-[(4-methoxyphenyl)amino]-2-oxoethoxy}-N-pyridin-3-ylbenzamide methyl 4-{2-[(4-methoxyphenyl)amino]-2-oxoethoxy}benzoate 4-{2-[(4-methoxyphenyl)amino]-2-oxoethoxy}benzoic acid 4-{2-[(4-methoxyphenyl)amino]-2-oxoethoxy}benzoyl chloride 2-(4-{2-[(4-fluorophenyl)amino]-2-oxoethyl}phenoxy}-N-2-methoxyphenyl)acetamide 2-(4-{2-[(4-methoxyphenyl)amino)-2-oxoethoxy}phenyl}-N-4-morpholin-4-ylphenyl)-acetamide 2-[4-(2-anilino-2-oxoethyl)phenoxy]-N-(4-methoxyphenyl)acetamide 2-(4-{2-[(4-methoxyphenyl)amino]-2-oxoethoxy}phenyl)-N-pyridin-3-ylethanamide ethyl 4-{2-[(4-methoxyphenylamino]-2-oxoethoxy}benzoate
N-[3-(1h-imidazol-1-yl)propyl]-4-{2-[(methoxyphenyl)amino]-2-oxoethoxy}benzamide methyl 4-{2-[(4-dimethylaminophenyl)amino]-2-oxoethoxy}benzoate methyl 4-(2-[(4-N-acetylaminophenyl)amino]-2-oxoethoxy}benzoate 4-(2-oxo-2-{[4-(trifluoromethoxy)phenyl]amino}ethoxy)benzamide 2-(4-cyanophenoxy-N-[4-(trifluoromethoxy)phenyl]acetamide 4-{2-[(4-fluorophenyl)amino]-2-oxoethoxy}benzamide 4-{2-[(4-Nitrophenyl)amino]-2-oxoethoxy}(benzamide methyl 4-{2-[(3-chlorophenyl)amino]-2-oxoethoxy}benzoate methyl 4-{2-[(4-fluorophenyl)amino]-2-oxoethoxy}benzoate methyl 4-(1-{[(4-fluorophenyl)amino]carbonyl}propoxy)benzoate and also the addition salts thereof.

14. Process according to claim 10, which is performed in basic medium, in a suitable solvent, with stirring and at reflux.

15. Process according to claim 14, for which the base is chosen from sodium hydroxide, potassium hydroxide and potassium carbonate.

16. Process according to claim 14, for which the solvent is chosen from amides.

17. Process according to claim 14, which is performed at between 70 and 160° C.

18. Process according to claim 10, wherein compound (I) is proposed using compounds of formula III and IV: 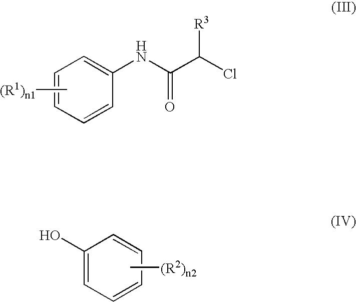
in which R′, R², R³, n1 and n2 are as defined in claim 10, and for which the process is performed in basic medium, in a suitable solvent, with stirring and at reflux.
The compound of the general formula (II) is obtained isolation of the intermediate compound (1) formed.

19. Use of the compounds of the general formula (I) 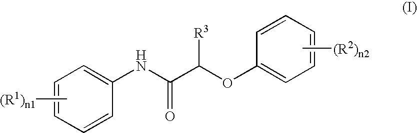
for the preparation of compounds of the general formula (II): 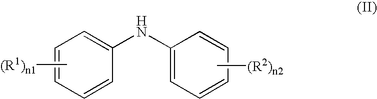
in which R′, R², R³, n1 and n2 are as defined in claim 10.

20. Process for the preparation of the compounds of the general formula (I) 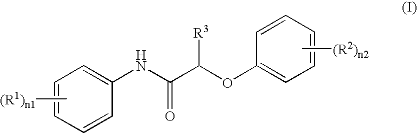
characterized in that the compounds of the general formulae (III) and (IV) are used: 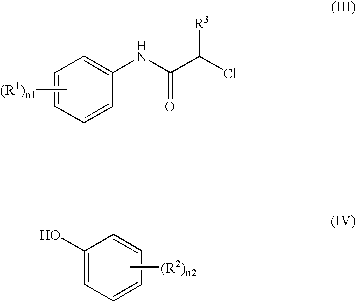
in which R′, R², R³, n1 and n2 are as defined in claim 10, and for which the process is performed in basic medium, in a suitable solvent, with stirring and at reflux.

21. Process according to claim 20, for which the base is chosen from sodium hydroxide, potassium hydroxide and potassium carbonate.

22. Process according to claim 21, for which the solvent is chosen from amides.

23. Process according to claim 21, which is performed at a temperature of between room temperature and 130° C.

24. Process according to claim 21, for which the compound of the general formula (III) is obtained by using the compounds of the general formulae (V) and (VI):

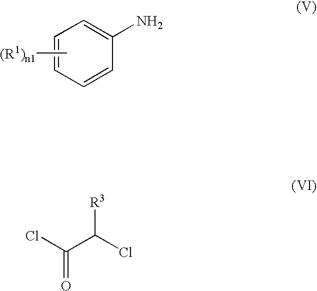

25. Compounds of the general formula (I) 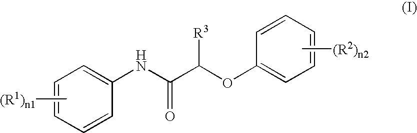
in which R¹, R², R³, n1 and n2 are as defined in claim 10, which can be obtained via the process according to claim 10.
